(19) United States
(12) Patent Application Publication
(10) Pub. No.: US 2021/0307995 A1
(43) Pub. Date: Oct. 7, 2021

(54) KNEADING MASSAGE MACHINE CORE

(71) Applicant: Xiamen Emoka Health Science & Technology Co., Ltd., Fujian (CN)

(72) Inventor: Guanghua ZHOU, Fujian (CN)

(73) Assignee: Xiamen Emoka Health Science & Technology Co., Ltd., Fujian (CN)

(21) Appl. No.: 17/015,005

(22) Filed: Sep. 8, 2020

(30) Foreign Application Priority Data
Apr. 2, 2020 (CN) 202020464588.7

(51) Int. Cl. A61H 7/00; A61N 5/06

(52) U.S. Cl. CPC A61H 7/007; A61N 5/0625; A61H 2201/169; A61H 2201/1671; A61H 2201/1436; A61H 2201/0207

(57) ABSTRACT

A kneading massage machine core includes a shell, a motor, transmission mechanisms, a printed circuit board (PCB), heating lamp panels, lamp panel fixing pieces and kneading massage heads. The motor, the transmission mechanisms and the PCB are arranged in the shell. The transmission mechanisms are connected to the motor and the kneading massage heads, the heating lamp panels are connected to the PCB, the kneading massage heads are connected to the transmission mechanisms and positioned on a top portion of the shell, and the heating lamp panels are installed on the lamp panel fixing pieces. The lamp panel fixing pieces are fixed on a top surface of the shell and correspond to the kneading massage heads, and a plurality of heat emission holes are formed in each of the kneading massage head.

### CROSS-REFERENCE TO RELATED APPLICATION

This application claims the priority benefit of China application serial no. 202020464588.7, filed on Apr. 2, 2020. The entirety of the above-mentioned patent application is hereby incorporated by reference herein and made a part of this specification.

### BACKGROUND

### Technical Field

The present disclosure relates to the technical field of massagers, in particular to a kneading massage machine core.

### Description of Related Art

At present, various kneading massagers appear on the market, so that convenience is brought to work and life of people, and the symptoms that people are prone to feel fatigue in hands, neck, cervical vertebra, feet and waist in life and work are effectively relieved. Chinese publication number CN202020649U discloses a kneading massage machine core which is composed of a motor, a motor shell, worms, primary gears, secondary gears, friction discs, massage heads, carbon brushes, a circuit box, transmission shafts, lamp panels, metal discs, circuit boards and carbon brush covers. When the friction discs rotate, the carbon brushes arranged in the massage discs are in contact with the metal discs arranged on the massage discs. Under the condition that the metal discs are powered on, the massage heads can also be electrified. An infrared lamp may be additionally arranged in each massage head according to needs, so that the massage heads can emit heat, and a better massage effect can be achieved.

The massage heads of the disclosed kneading massage machine core achieve a heating function through the infrared lamps on the massage heads, the massage heads are in contact with the metal discs through the carbon brushes in the massage discs, and the friction heads are electrified by powering on the metal discs. The massage machine core with such structure has the following defects. Firstly, the kneading massage heads adopt a split structure, the process is complex, the assembly time needs to be prolonged, and the production efficiency needs to be reduced. Secondly, the heating mode of the kneading massage machine core is that electricity is transmitted to the infrared lamps in the massage heads through the contact between the metal discs and the carbon brushes, so that the infrared lamps achieve a heating function, the heating mode is complex in structure and tedious in processing procedure, the heating effect is not good since the infrared lamps are arranged in the massage heads, and improvement is really needed.

### SUMMARY

The present disclosure aims at overcoming deficiencies of the prior art, and providing kneading massage heads and a kneading massage machine core which are relatively simple in structure and technology and good in heating effect.

In order to achieve the above-mentioned purpose, the technical solution of the present disclosure is as follows.

A kneading massage machine core includes a shell, a motor, transmission mechanisms, a printed circuit board (PCB), heating lamp panels, lamp panel fixing pieces and kneading massage heads. The motor, the transmission mechanisms and the PCB are arranged in the shell, the transmission mechanisms are connected to the motor and the kneading massage heads, the heating lamp panels are connected to the PCB, the kneading massage heads are connected to the transmission mechanisms and positioned on a top portion of the shell, the heating lamp panels are installed on the lamp panel fixing pieces, the lamp panel fixing pieces are fixed on a top surface of the shell and correspond to the kneading massage heads, and a plurality of heat emission holes are formed in each of the kneading massage heads.

After adopting the above-mentioned solution, according to the kneading massage machine core, the motor drives the transmission mechanisms to rotate, the transmission mechanisms drive the kneading massage heads connected to the transmission mechanisms to rotate, so as to achieve kneading massage. The heating lamp panels are fixed on the lamp panel fixing pieces that are fixed on a top surface of the shell, when the kneading massage heads rotate, the heating lamp panels will not rotate along with the kneading massage heads, instead, the heating lamp panels are directly connected to the PCB through wires to carry out electrical heating, and heat generated by the heating lamp panels is directly emitted through the heat emission holes in the kneading massage heads to achieve heating massage. Compared with an existing kneading massage machine core mechanism adopting split-type kneading massage heads that need to be assembled, the kneading massage machine core provided by the present disclosure adopts the integrated kneading massage heads that are simpler in structure and do not need to be assembled, the heat dissipation holes are directly formed in the massage heads, direct heating is achieved by the heating lamp panels, and the heating effect is better. Moreover, compared with the complex process that the existing massage machine core transmits electricity to the heating lamp panels through conductive plates and carbon brushes for heating, the heating mode of the kneading massage machine core provided by the present disclosure is that the heating lamp panels are directly connected to the circuit board through the wires for carrying out electrical heating, the power supply mode of the heating lamp panels is simple, heat is directly emitted through the heat emission holes formed in the kneading massage heads for direct heating, and the heating effect is good.

Furthermore, the shell includes an upper shell and a lower shell that matched with each other, a motor cavity for allowing the motor to be installed therein is defined by middle parts of the upper shell and the lower shell, the transmission mechanisms are arranged on two sides of the motor cavity, the motor is fixedly installed in the motor cavity of the shell, and output shafts are arranged at two ends of the motor respectively.

Furthermore, the transmission mechanisms include secondary gears meshed with the two output shafts of the motor and driving gears meshed with the secondary gears, rotation shafts of the driving gears protrude to a top portion of the shell and are fixed to and matched with the kneading massage heads, each of the secondary gears includes a spur gear and a helical gear which are fixed on a same rotation shaft, and the helical gears are meshed with the output shafts of the motor. When the motor is in work, the two output shafts are driven to rotate, the two output shafts will drive the secondary gears meshed with the output shafts to rotate when the two output shafts rotate, the spur gears of the secondary gears will drive the driving gears meshed with the secondary gears to rotate when the spur gears of the secondary gears rotate, and thus the kneading massage heads fixed on the rotation shafts of the driving gears are enabled to rotate to achieve kneading massage.

Furthermore, the PCB is fixed on the lower shell of the shell, and covered by an upper cover. The heating lamp panels are connected to the PCB through the wires. The lamp panel fixing pieces are locked on atop surface of the upper shell.

Furthermore, far infrared lamps are disposed on each of the heating lamp panels, and positions and number correspond to those of the heat emission holes in each of the kneading massage heads.

Furthermore, the kneading massage heads are detachably matched with the transmission mechanisms. When the kneading massage machine core is in use, different massage effects can be achieved by replacing different massage heads according to actual needs of a user.

Furthermore, the kneading massage heads are further provided with elastic massage heads, each of the elastic massage heads include an elastic connector and a massage bulge, one end of the elastic connector is fixed on a respective one of the kneading massage heads, and the other end of the elastic connectors is connected to the massage bulge. When the kneading massage machine core is in use, the elastic connectors can telescopically deform according to different stress conditions, so as to self-adapt to to-be-massaged parts of the user and improve massage experience.

### BRIEF DESCRIPTION OF THE DRAWINGS

FIG. 1 is a schematic diagram of a structure of a first preferred embodiment of a kneading massage machine core provided by the present disclosure.

FIG. 2 is a schematic diagram of a structure of a second preferred embodiment of the kneading massage machine core provided by the present disclosure.

### DESCRIPTION OF THE EMBODIMENTS

In order to further explain the technical solution of the present disclosure, the present disclosure is set forth in detail below by specific embodiments.

In description of the present disclosure, it should be understood that the terms “center”, “longitudinal”, “lateral”, “upper”, “lower”, “front”, “rear”, “left”, “right”, “vertical”, “horizontal”, “top”, “bottom”, “inner”, “outer” and the like indicate orientations or positional relationships that are based on the orientations or positional relationships shown in accompanying drawings, are only for the convenience of describing the present disclosure and simplifying the description, rather than indicating or implying that the device or element referred to must have the specific orientation and must be constructed and operated in the specific orientation, and thus cannot be understood as a limitation on the present disclosure. In the description of the present disclosure, unless otherwise indicated, the meaning of “a plurality of” is two or more.

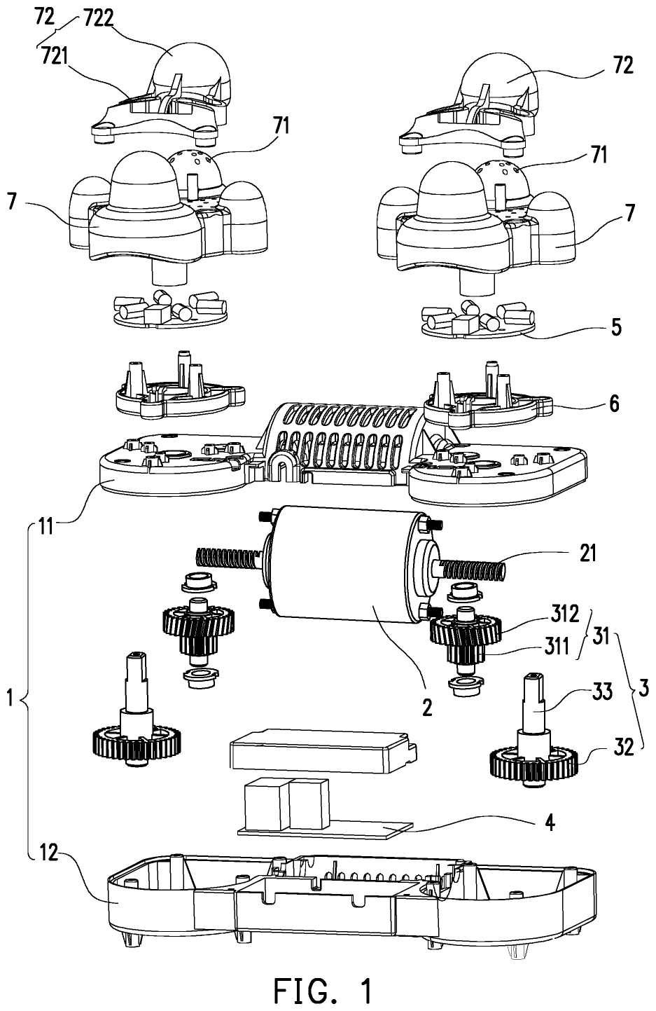

As shown in FIG. 1, a first preferred embodiment of a kneading massage machine core provided by the present disclosure includes a shell 1, a motor 2, transmission mechanisms 3, a printed circuit board (PCB) 4, heating lamp panels 5, lamp panel fixing pieces 6 and kneading massage heads 7. The motor 2, the transmission mechanisms 3 and the PCB 4 are arranged in the shell 1, the transmission mechanisms 3 are connected to the motor 2 and the kneading massage heads 7, the heating lamp panels 5 are connected to the PCB 4, the kneading massage heads 7 are connected to the transmission mechanisms 3 and positioned on a top portion of the shell 1, the heating lamp panels 5 are installed on the lamp panel fixing pieces 6, the lamp panel fixing pieces 6 are fixed on a top surface of the shell 1 and positioned below the kneading massage heads 7, and a plurality of heat emission holes 71 are formed in each of the kneading massage heads 7.

The working principle of the present disclosure is as follows. The motor 2 drives the transmission mechanisms 3 to rotate, the transmission mechanisms 3 drive the kneading massage heads 7 connected to the transmission mechanisms 3 to rotate, so as to achieve kneading massage. The heating lamp panels 5 are fixed on the lamp panel fixing pieces 6 that are fixed on the top surface of the shell 1, when the kneading massage heads 7 rotate, the heating lamp panels 5 will not rotate along with the kneading massage heads 7, instead, the heating lamp panels 5 are directly connected to the PCB 4 through the wires to carry out electrical heating, and heat generated by the heating lamp panels 5 is directly emitted through the heat emission holes 71 in the kneading massage heads 7 to achieve heating massage.

The shell 1 includes an upper shell 11 and a lower shell 12 that can be matched with each other, a motor cavity for allowing the motor 2 to be installed therein is defined by the middle parts of the upper shell 11 and the lower shell 12, and the transmission mechanisms 3 are arranged on two sides of the motor cavity. The motor 2 is fixedly installed in the motor cavity of the shell 1, output shafts 21 are arranged at two ends of the motor 2 respectively. The transmission mechanisms 3 respectively include secondary gears 31 mutually meshed with the two output shafts 21 of the motor 2 and driving gears 32 meshed with the secondary gears 31. Rotation shafts 33 of the driving gears 32 protrude to the top of the shell 1 and are fixed to and matched with the kneading massage heads 7, respectively. Each of the secondary gears 31 includes a spur gear 311 and a helical gear 312 which are fixed on the same rotation shaft, and the helical gears 312 are meshed with the output shafts 21 of the motor 2. When the motor 2 is in work, the two output shafts 21 are driven to rotate. The two output shafts 21 will drive the secondary gears 31 meshed with the output shafts 21 to rotate when the two output shafts 21 rotate, the spur gears 311 of the secondary gears 31 will drive the driving gears 32 meshed with the secondary gears 31 to rotate when the spur gears 311 of the secondary gears 31 rotate, and thus the kneading massage heads 7 fixed on the rotation shafts 33 of the driving gears 32 are enabled to rotate to achieve kneading massage.

The PCB 4 is fixed on the lower shell 12 of the shell 1, and covered by an upper cover 41. The heating lamp panels 5 are connected to the PCB 4 through the wires. The lamp panel fixing pieces 6 are locked on the top surface of the upper shell 11. Far infrared lamps are arranged on each of the heating lamp panels 5. The positions and number of the far infrared lamps correspond to those of the heat emission holes 71 in each of the kneading massage heads 7.

In order to enable the kneading massage heads 7 of the present disclosure to have different massage effects, the kneading massage heads 7 are detachably matched with the transmission mechanisms 3. As shown in FIG. 1, the tops of the rotation shafts 33 of the transmission mechanisms 3 are detachably matched with the bottoms of the massage heads, so that the kneading massage heads 7 of the present disclosure may be replaced according to actual needs of users.

In a first embodiment, in order to enable the kneading massage heads 7 of the present disclosure to adapt to the users with different body types, the kneading massage heads 7 are also provided with elastic massage heads 72. Each of the elastic massage heads 72 include an elastic connector 721 and a massage bulge 722, one end of the elastic connector 721 is fixed on the kneading massage heads 7, and the other end of the elastic connector 721 is connected to the massage bulges 722. When the kneading massage machine core is in use, the elastic connectors 721 can telescopically deform according to different stress conditions, so as to self-adapt to to-be-massaged parts of the user and improve massage experience.

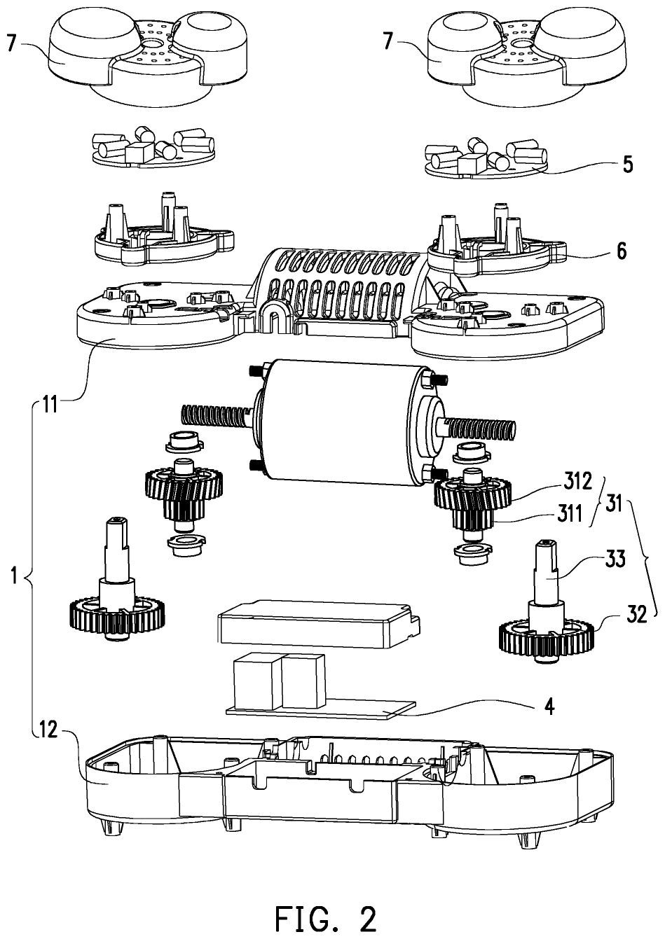

As shown in FIG. 2, a second preferred embodiment of the kneading massage machine core provided by the present disclosure differs from the first embodiment in that the structure of the massage heads 7 is different, and aims to show that different massage effects can be achieved by replacing different kneading massage heads 7. The massage heads of the first embodiment can be suitable for the users of different body types, and when the massage heads are used for kneading, certain elasticity is provided, and a better comfort degree is achieved. The massage heads of the second embodiment are simple in structure, still can achieve the kneading massage effect, and are relatively low in cost. Besides the massage heads shown in the first embodiment and the second embodiment, the massage heads of other structures may be adopted for replacing, and different massage effects can be achieved by assembling different massage heads.

The above-described embodiments and drawings are not intended to limit the form and style of products of the present disclosure, and any suitable variations or modifications made thereto by one of ordinary skill in the art should be deemed to not depart from the scope of the patent of the present disclosure.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A kneading massage machine core, comprising a shell, a motor, transmission mechanisms, a printed circuit board (PCB), heating lamp panels, lamp panel fixing pieces and kneading massage heads, wherein the motor, the transmission mechanisms and the PCB are arranged in the shell, the transmission mechanisms are connected to the motor and the kneading massage heads, the heating lamp panels are connected to the PCB, the kneading massage heads are connected to the transmission mechanisms and positioned on a top portion of the shell, the heating lamp panels are installed on the lamp panel fixing pieces, the lamp panel fixing pieces are fixed on a top surface of the shell and correspond to the kneading massage heads, and a plurality of heat emission holes are formed in each of the kneading massage heads.

2. The kneading massage machine core according to claim 1, wherein the shell comprises an upper shell and a lower shell matched with each other, a motor cavity for allowing the motor to be installed therein is defined by middle parts of the upper shell and the lower shell, the transmission mechanisms are arranged on two sides of the motor cavity, the motor is fixedly arranged in the motor cavity of the shell, and output shafts are arranged at two ends of the motor respectively.

3. The kneading massage machine core according to claim 2, wherein the transmission mechanisms comprise secondary gears meshed with the two output shafts of the motor and driving gears meshed with the secondary gears, rotation shafts of the driving gears protrude to the top portion of the shell and are fixed to and matched with the kneading massage heads, each of the secondary gears comprises a spur gear and a helical gear which are fixed on a same rotation shaft, and the helical gears are meshed with the output shafts of the motor.

4. The kneading massage machine core according to claim 2, wherein the PCB is fixed on the lower shell of the shell and covered by an upper cover, the heating lamp panels are connected to the PCB via wires, and the lamp panel fixing pieces are locked on a top surface of the upper shell.

5. The kneading massage machine core according to claim 1, wherein far infrared lamps are disposed on each of the heating lamp panels, and positions and number of the far infrared lamps correspond to those of the heat emission holes in each of the kneading massage heads.

6. The kneading massage machine core according to claim 1, wherein the kneading massage heads and the transmission mechanisms are detachably matched together.

7. The kneading massage machine core according to claim 1, wherein the kneading massage heads are further provided with elastic massage heads, each of the elastic massage heads comprises an elastic connector and a massage bulge, one end of the elastic connector is fixed on a respective one of the kneading massage heads, and the other end of the elastic connector is connected to the massage bulge.
